(19) United States
(12) Patent Application Publication
(10) Pub. No.: US 2002/0165196 A1
(43) Pub. Date: Nov. 7, 2002

(54) Oligonucleotide inhibitors of cancer cell proliferation

(76) Inventor: Eric Wickstrom, Philadelphia, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 10/141,263

(22) Filed: May 7, 2002

Related U.S. Application Data
(60) Provisional application No. 60/289,166, filed on May 7, 2001.

(51) Int. Cl. A61K 48/00; C07H 21/04

(52) U.S. Cl. 514/44; 536/23.2

(57) ABSTRACT

The K-RAS oncogene is a member of the highly conserved RAS gene family, whose protein products are believed to play a significant role in signal transduction and the regulation of cellular proliferation. Mutation-activated K-RAS is found in 30-50% of both advanced and early stage ovarian cancers. The present invention relates to a method for modulating mutation-activated K-RAS expression in ovarian, colon, lung, thyroid, prostate, skin, and hematologic cancer cells by administering an effective amount of an oligonucleotide targeted against a portion of mRNA for human K-RAS. Oligonucleotides are provided that are specifically hybridizable with mRNA encoding mutation-activated human K-RAS. Such oligonucleotides can be used for therapeutics and diagnostics as well as for research purposes. The present invention further encompasses pharmaceutical compositions comprising the antisense oligonucleotides of the invention.

### CONTINUING APPLICATION DATA

This application claims priority under 35 U.S.C. §119 based upon U.S. Provisional Application No. 60/289,166 filed May 7, 2001.

### GOVERNMENT RIGHTS TO THE INVENTION

This invention was made with government support under grant U01-CA60139 awarded by the National Cancer Institute. The government has certain rights in the invention.

### FIELD OF THE INVENTION

The present invention relates to the fields of molecular biology and genetics and to a method of treating or preventing cancer and, more particularly, to the modulation of K-RAS gene expression in cancerous cells.

### BACKGROUND OF THE INVENTION

Cancer results from a multistep process of oncogene activation and/or suppressor gene inactivation. (Bishop, Cell 64:235-248, 1991). Alterations in cellular genes that directly or indirectly control cell growth and differentiation are considered to be the main cause of cancer. There are some thirty families of genes, called oncogenes, that are implicated in human tumor formation. Members of one such family, the RAS gene family, are carried in a broad range of eukaryotes and are frequently found to be mutated in human tumors. Humans carry three functional RAS oncogenes, H-RAS, K-RAS, and N-RAS, coding for 21 kDa proteins 188-189 amino acids long. (Lowy & Willumsen, Annu. Rev. Biochem. 62:851-891, 1993). K-RAS, H-RAS, and N-RAS have been detected in more human tumor types and at higher frequencies than any other oncogenes. (Bishop, Cell 64:235-248, 1991).

In their normal state, proteins produced by the RAS genes are thought to be involved in normal cell growth and maturation. Mutation of the RAS gene, causing an amino acid alteration at one of three critical positions in the protein product, results in conversion to a form that is implicated in tumor formation. Over 90% of pancreatic adenocarcinomas, about 50% of prostate cancers, about 50% of adenomas and adenocarcinomas of the colon, about 50% of adenocarcinomas of the lung, about 50% of carcinomas of the thyroid, about 25% of melanomas, and a large fraction of malignancies of the blood, such as acute myeloid leukemia and myelodysplastic syndrome, have been found to contain activated RAS oncogenes. Overall, at least one-third of human tumors have a mutation in one of the three RAS genes. In particular, the K-RAS oncogene is activated in 30-50% of both advanced and early stage ovarian cancers with mutations in the 12^{th}, or occasionally the 13^{th}, codon. (Almoguera et al., Cell 53:549-554, 1988; Shukla et al. Oncogene Res. 5:121-127, 1989; Mok et al., Cancer Res. 53:1489-1492, 1993; Morita et al., Pathol. Int. 50:219-223, 2000; Suzuki et al., Cancer Genet. Cytogenet. 118:132-135, 2000).

Invasive ovarian cancer strikes approximately 23,100 women in the United States each year, with the majority of patients presenting with advanced stage disease. It accounts for 4% of all cancers among women and ranks second among gynecologic cancers, fourth among all cancers. Ovarian cancer causes more deaths than any cancer of the female reproductive system. The 5-year survival rate is 79% if the disease is localized to the region of the ovary, but is only 28% for patients with distant metastases at the time of diagnosis. Mortality rates for ovarian cancer are static despite recent advances in the treatment of advanced disease, the screening for early cancer, and the fundamental knowledge about the molecular and cellular events that underlie this disease.

Many varieties of Ras proteins have been found. These proteins are very homologous in amino acid sequence, differing primarily at their C termini. The K-RAS oncogene codes for an evolutionarily conserved G-protein, K-Ras p21, which binds guanine nucleotides with high affinity and hydrolyzes GTP with low catalytic efficiency. This protein is associated with the inner surface of the plasma membrane and appears to play a fundamental role in basic cellular regulatory functions relating to the transduction of extracellular signals across plasma membranes. (Lowy & Willumsen, Annu. Rev. Biochem. 62:851-891, 1993). Specifically, the Ras:GDP complex receives a signal from an upstream element (i.e., an activated membrane bound receptor) and the GDP is exchanged for GTP, thereby converting the inactive Ras:GDP complex to the active Ras:GTP complex. (Downward et al., Proc. Natl. Acad. Sci. USA 87:5998-6002, 1990). The Ras:GTP complex is able to transmit the signal downstream to an appropriate target. The active Ras:GTP complex is converted to the inactive GDP complex by hydrolysis of the GTP to GDP.

Mammalian RAS genes acquire transformation-inducing properties by single point mutations within their coding sequences. Mutations in naturally occurring RAS oncogenes have been localized to codons 12, 13, and 61. The Ras protein itself possesses intrinsic GTPase activity; however, in vivo this intrinsic activity is very slow unless enhanced by GAP (GTPase-activating protein). The main biochemical difference between oncogenic Ras proteins with mutations in codon 12, 13, or 61 and wild-type p21 is the ability of GAP to induce GTP hydrolysis in the active Ras:GTP complex. The GAP-induced hydrolysis can be as much as 1000 times greater in the wild-type Ras than in these mutant forms of Ras. (Gibbs et al., Proc. Natl. Acad. Sci. USA 85:5026-5030, 1988). These mutant forms remain in the active GTP form much longer than the wild-type, and presumably, the continual transmission of a signal by the mutant forms is responsible for their oncogenic properties. It, therefore, is believed that inhibition of RAS expression is useful in treatment and/or prevention of malignant conditions, i.e., cancer and other hyperproliferative conditions.

Inhibition of K-Ras protein appears to be part of the mechanism of antiproliferation of paclitaxel, a natural product that binds to the microtubules along which K-Ras may traverse. (Thissen et al., J. Biol. Chem. 272:30362-30370, 1997). Unfortunately, paclitaxel displays strong dose-limiting toxicity that limits its efficacy. (Seidman, Semin. Oncol. 26:(3 Suppl 8), 14-20, 1999). Prevention of K-Ras post-translational farnesylation by farnesyltransferase inhibitors also has been associated with inhibition of the growth of Ras-dependent tumors in immunocompromised mice. (Prendergast et al., Mol. Cell. Biol. 14:4193-202, 1994). Rho, however, appears to be the principal target of farnesyltransferase inhibitors, rather than the intended Ras. (Prendergast, Curr. Opin. Cell Biol. 12:166-173, 2000). Despite determined efforts by academic and industrial scientists, to date no specific inhibitor of activated K-Ras protein has been identified.

Reducing the level of K-RAS gene expression might inhibit proliferation or reverse transformation in malignant cells transformed by mutated K-RAS. (Georges et al., Cancer Res. 53:1743-1746, 1993; Mukhopadhyay et al., Cancer Res. 51:1744-1748, 1991; Kashani-Sabet et al., Cancer Res. 54:900-902, 1994; Aoki et al., Cancer Res. 55:3810-3816, 1995; Kawada et al., Biochem Biophys Res Commun 231:735-737, 1997; Kita et al., Int J Cancer 80:553-558, 1999; Okada et al., Proc Natl Acad Sci USA 95:3609-3614, 1998; Wickstrom & Tyson, in Chadwick, D. J., and Cardew, G., eds., Oligonucleotides as Therapeutic Agents, Ciba Foundation Symposium 209, Wiley, Chichester, 124-141, 1997). Feramisco et al. (Nature 314:639-642, 1985), demonstrated that if cells transformed to a malignant state with an activated H-RAS gene are microinjected with antibody that binds to the H-Ras protein product of the H-RAS gene, the cells slow their rate of proliferation and adopt a more normal appearance. Consequently, there is need for compositions of matter that are able to modulate the expression of activated RAS oncogenes, and in particular, compositions that specifically modulate the expression of mutation-activated K-Ras protein.

Molecular strategies are being developed to downregulate unwanted gene expression, including oncogene expression. One such strategy involves inhibiting gene expression with small oligonucleotides complementary in sequence to, and thus able to specifically hybridize with, the mRNA transcript of a target gene. Antisense DNAs were first conceived as alkylating complementary oligodeoxynucleotides directed against naturally occurring nucleic acids (Belikova, et al., Tetrahedron Lett. 37:3557-3562, 1967). Zamecnik and Stephenson were the first to propose the use of synthetic antisense oligonucleotides for therapeutic purposes. (Zamecnik & Stephenson, Proc. Natl. Acad. Sci. USA, 75:285-289, 1978; Zamecnik & Stephenson, Proc. Natl. Acad. Sci. USA, 75:280-284, 1978). They reported that the use of an oligonucleotide 13-mer complementary to the RNA of Rous sarcoma virus inhibited the growth of the virus in cell culture. Since then, numerous other studies have been published manifesting the in vitro efficacy of antisense oligonucleotide inhibition of viral growth, e.g., vesicular stomatitis viruses (Leonetti et al., Gene, 72:323; 1988; herpes simplex viruses (Smith et al., Proc. Natl. Acad. Sci. U.S.A. 83:2787, 1986), and influenza virus (Serial et al., Nucleic Acids Res. 15:9909, 1987).

Antisense oligonucleotides that target various oncogenes or proto-oncogenes have been proposed as anti-cancer agents. By binding to the complementary nucleic acid sequence in RNA, antisense oligonucleotides are able to inhibit splicing and translation of RNA. In this way, antisense oligonucleotides are able to inhibit protein expression. Heikkila et al. (Nature, 328:445-449, 1987) showed that antisense oligonucleotides hybridizing specifically with mRNA transcripts of the oncogene c-MYC, when added to normal human lymphocytes stimulated with phorbol myristic acetate, inhibited not only expression of the c-Myc protein product of the c-MYC oncogene but also inhibited proliferation. Wickstrom et al. (Proc. Natl. Acad. Sci. USA 85:1028-1032, 1988) showed that expression of the protein product of the c-MYC oncogene as well as proliferation of HL60 cultured leukemic cells were inhibited by antisense oligonucleotides hybridizing specifically with c-MYC mRNA. Anfossi et al. (Proc. Natl. Acad. Sci. USA 86:3379-3383, 1989) showed that antisense oligonucleotides specifically hybridizing with mRNA transcripts of the c-MYB oncogene inhibited proliferation of human myeloid leukemia cell lines.

The present invention relates to compositions and methods for modulating the expression of mutation-activated K-Ras. More specifically, the present invention provides a method for the treatment of cancers associated with K-RAS expression involving antisense oligonucleotides that are targeted to mRNA encoding human K-RAS and are capable of inhibiting K-RAS expression. In particular, the present invention provides a method for the treatment for ovarian, colon, lung, thyroid, prostate, skin, and hematologic cancers associated with K-RAS expression.

Definitions

The term “oncogene” as used herein means a human gene in a host cell that is responsible, in whole or in part, for the neoplastic transformation of the host cell.

As used herein the term “antisense oligonucleotide specific for” a targeted oncogene means an oligonucleotide capable of forming a stable duplex with a portion of an mRNA transcript of a targeted oncogene.

“Targeting” an oligonucleotide to a chosen nucleic acid target, in the context of this invention, is a multistep process. The process usually begins with identifying a nucleic acid sequence whose function is to be modulated. This may be, for example, a cellular gene (or mRNA made from the gene) whose expression is associated with a particular disease state, or a foreign nucleic acid from an infectious agent. In the present invention, the target is a nucleic acid encoding K-RAS; in other words, the K-RAS gene or mRNA expressed from the K-RAS gene. The targeting process also includes determination of a site or sites within the nucleic acid sequence for the oligonucleotide interaction to occur such that the desired effect—modulation of gene expression—will result. Once the target site or sites have been identified, oligonucleotides are chosen which are sufficiently complementary to the target, i.e., hybridize sufficiently well and with sufficient specificity, to give the desired modulation.

As used herein “hybridization” means hydrogen bonding, also known as Watson-Crick base pairing, or the like (such as Hoogsteen or reverse Hoogsteen types of base pairing) between complementary bases, usually on opposite nucleic acid strands or two regions of a nucleic acid strand. Guanine and cytosine are examples of complementary bases that are known to form three hydrogen bonds between them. Adenine and thymine are examples of complementary bases which form two hydrogen bonds between them. “Specifically hybridizable” and “complementary” are terms that are used to indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

The term “oligonucleotide” as used herein includes linear oligomers of natural or modified monomers or linkages, including deoxyribonucleosides, ribonucleosides, α-anomeric forms thereof, polyamide nucleic acids, and the like, capable of specifically binding to a target polynucleotide by way of a regular pattern of monomer-to-monomer interactions, such as Watson-Crick type of base pairing, Hoogsteen or reverse Hoogsteen types of base pairing, or the like. Usually, monomers are linked by phosphodiester bonds or analogs thereof to form oligonucleotides ranging in size from a few monomeric units, e.g., 3-4, to several hundreds of monomeric units. Analogs of phosphodiester linkages include: phosphorothioate, phosphorodithioate, phosphoroselenoate, phosphorodiselenoate, phosphoroanilothioate, phosphoranilidate, phosphoramidate, and the like, as more fully described below.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs (John Wiley, New York, 1980). Such analogs include synthetic nucleosides designed to enhance binding properties, e.g., duplex stability, specificity, or the like.

The term “phosphorothioate oligonucleotide” means an oligonucleotide wherein one or more of the internucleotide linkages is a phosphorothioate group as opposed to the phosphodiester group, which is characteristic of unmodified oligonucleotides.

The term “alkylphosphonate oligonucleoside” as used herein means an oligonucleotide wherein one or more of the internucleotide linkages is an alkylphosphonate group.

The term “modified oligonucleotide” means an oligonucleotide containing one or more modified monomers and/or linkages to enhance the stability or uptake of the oligonucleotide (supra).

The term “modulation” as used herein means either inhibition or stimulation.

“Stability,” in reference to duplex formation, roughly means how tightly an antisense oligonucleotide binds to its intended target sequence; more precisely, it means the free energy of formation of the duplex under physiological conditions. Melting temperature under a standard set of conditions (infra) is a convenient measure of duplex stability. Preferably, antisense oligonucleotides of the invention are selected that have melting temperatures of at least 50° C. under the standard conditions set forth below; thus, under physiological conditions and the preferred concentrations, duplex formation will be substantially favored over the state in which the antisense oligonucleotide and its target are dissociated. It is understood that a stable duplex may in some embodiments include mismatches between base pairs. Preferably, antisense oligonucleotides of the invention are perfectly matched with their target polynucleotides.

### BRIEF DESCRIPTION OF THE DRAWINGS

FIG. 1. Effect of intraperitoneal oligonucleotides, 1 mg every other day for 14 days, on survival of nude mice bearing intraperitoneal human OVCAR5 ovarian cancer cells. Three independent trials with 10 mice per treatment group are summed.

FIG. 2. Oligonucleotide backbone derivatives.

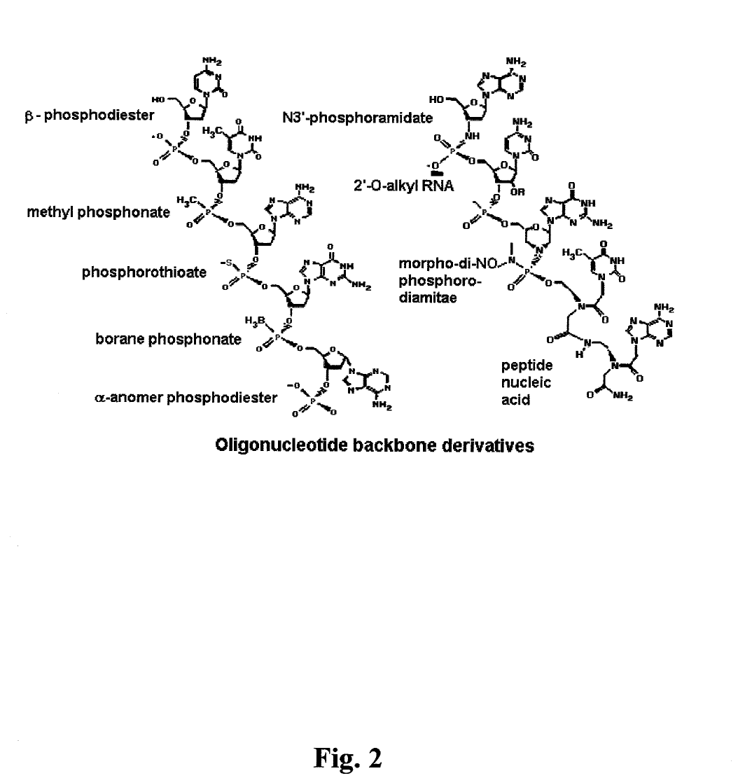

### DETAILED DESCRIPTION OF THE INVENTION

Methods

Target Polynucleotide

The target polynucleotide of the present invention comprises an mRNA transcript of K-RAS, specifically a mutant form of K-RAS. Oligonucleotides complementary to and hybridizable with the specified portions of the mRNA transcript are, in principle, effective for modulating translation, and capable of inducing the effects herein described. The functions of mRNA to be interfered with include, but are not limited to, translocation of the RNA to the site for protein translation, actual translation of protein from the RNA, splicing or maturation of the RNA, and independent catalytic activity that may be engaged in by the RNA. The overall effect of such interference with mRNA function is to cause interference with K-RAS protein expression.

In one embodiment of the invention, inhibition of K-RAS gene expression is the form of modulation. Modulation can be measured in ways that are routine in the art, for example by Northern blot assay of mRNA expression or Western blot assay of protein expression. Effects on cell proliferation or tumor cell growth also can be measured in ways that are known in the art.

In accordance with this invention, persons of ordinary skill in the art will understand that mRNA includes not only the coding region that carries the information to encode a protein using the three letter genetic code, including the translation start and stop codons, but also associated ribonucleotides that form a region known to such persons as the 5′-untranslated region, the 3′-untranslated region, the 5′ cap region, intron regions, and intron/exon or splice junction ribonucleotides. Thus, oligonucleotides may be formulated in accordance with this invention that are targeted wholly or in part to these associated ribonucleotides as well as to the coding ribonucleotides. In one embodiment of the invention, the portion of the mRNA that is the target polynucleotide is the 10 nucleotide sequence K-RAS mRNA from nucleotide 138-147 (SEQ. ID. NO. 5).

Oligonucleotide Selection

The present invention relates to a method for selectively modulating mutation-activated K-RAS expression in cancer cells by administering an effective amount of an oligonucleotide complementary to a portion of mRNA for human K-RAS. The oligonucleotides of the present invention specifically hybridize to mRNA transcribed from a mutant form of K-RAS. There is substantial guidance in the literature for selecting particular sequences for antisense oligonucleotides given a knowledge of the sequence of the target polynucleotide, see e.g., Daaka & Wickstrom, Oncogene Res. 5:267-275, 1990; Bacon & Wickstrom, Oncogene Res. 6:13-19, 1991; Wickstrom, Prospects for Antisense Nucleic Acid Therapy of Cancer and AIDS, Wiley-Liss, New York (1991); Crooke, Ann. Rev. Pharmacol. Toxicol. 32:329-376, 1992. Oligonucleotides are chosen that are sufficiently complementary to the specified portion of the target, i.e., hybridize sufficiently well, and with sufficient specificity, to give the desired modulation.

In general, the antisense oligonucleotides used in the practice of the present invention will have a sequence that is completely complementary to a selected portion of the target polynucleotide. Absolute complementarity, however, is not required, particularly in larger oligomers. Thus, reference herein to a “nucleotide sequence complementary to” a target polynucleotide does not necessarily mean a sequence having 100% complementarity with the target segment. In general, any oligonucleotide having sufficient complementarity to form a stable duplex with the target (e.g., an oncogene mRNA), that is an oligonucleotide that is “hybridizable,” is suitable. Stable duplex formation depends on the sequence and length of the hybridizing oligonucleotide and the degree of complementarity with the target polynucleotide. Generally, the larger the hybridizing oligomer, the more mismatches may be tolerated. More than two separated mismatches probably will not be tolerated for antisense oligomers of less than about 21 nucleotides. One skilled in the art may readily determine the degree of mismatching that may be tolerated between any given antisense oligomer and the target sequence based upon the melting temperature (Tm) and, therefore, the thermal stability of the resulting duplex.

The oligonucleotides used were the following: 5′-dAGTCGCCCCGCCGCA-3′ (NSC717139) (SEQ. ID. NO: 1); 5′-dAGTCGAAAAGCCGCA-3′ (NSC717140) (SEQ. ID. NO: 2); 5′-dGGTGCTCACTGCGGC-3′ (NSC717137) (SEQ. ID. NO: 3); and 5′-dGGTGCAGTGTGCGGC-3′ (NSC717138) (SEQ. ID. NO: 4). Additional oligonucleotides of the instant invention include the following: 5′-dGCCCCGCCGC-3′ (KRAS8) (SEQ. ID. NO: 6); and 5′-dGAAAAGCCGC-3′ (KRAS9) (SEQ. ID. NO: 7).

Thermal Stability

Preferably, the thermal stability of hybrids formed by the antisense oligonucleotides of the invention are determined by way of melting, or strand dissociation, curves. (Wickstrom & Tinoco, Biopolymers 13:2367-2383, 1974). The temperature of 50% strand dissociation is taken as the melting temperature, Tₘ, which, in turn, provides a convenient measure of stability. Tₘ measurements are typically carried out in a saline solution at neutral pH with target and antisense oligonucleotide concentrations at between about 1.0-2.0 μM. Typical conditions that yield physiological relevant measurements are as follows: 1.0 M NaCl (or 150 mM NaCl and 10 mM MgCl₂) in a 10 mM sodium phosphate buffer (pH 7.0) or in a 10 mM Tris-HCl buffer (pH 7.0). Data for melting curves typically are accumulated by heating a sample of the antisense oligonucleotide/target polynucleotide complex from 5-10° C. up to 80-90° C. As the temperature of the sample increases, absorbance of 260 nm light is monitored at 1° C. intervals, using e.g., a Cary (Australia) model 3E or a Hewlett-Packard (Palo Alto, Calif.) model HP 8459 UV/VIS spectrophotometer and model HP 89100A temperature controller, or like instruments. Such techniques provide a convenient means for measuring and comparing the binding strengths of antisense oligonucleotides of different lengths and compositions.

In one embodiment, the region of the oligonucleotide that is modified to increase K-RAS mRNA binding affinity comprises at least one 5′ or 3′ terminal nucleotide modified at the 2′ position of the sugar, most preferably a 2′-O-alkyl, 2′-O-alkyl-O-alkyl or 2′-fluoro-modified nucleotide. Such modifications are routinely incorporated into oligonucleotides and these chimeric or mixed backbone oligonucleotides have been shown to have a higher Tm (i.e., higher target binding affinity) than homogeneous 2′-oligodeoxynucleotides against a given target. The effect of such increased affinity is to greatly enhance antisense oligonucleotide inhibition of K-RAS gene expression.

### EXAMPLE 1

### Cell Culture

Cell Lines

The human OVCAR5 ovarian cancer cell line is derived from the ascites of an untreated female patient, an excellent model for terminal ovarian cancer ascites. (Louie, et al., Biochem. Pharmacol. 35:467-472, 1986). OVCAR5 cells display overexpression of both ERBB2 and 12^{th} codon mutated K-RAS. (NIH/NCI/DCTD/DTP, unpublished).

Antigen Levels

For each assay, the OVCAR5 cancer cells were grown in complete RPMI 1640 medium with 2 mM glutamine, pen/strep, and 10% fetal bovine serum, and maintained in log phase. Three days preceding the analysis of antisense inhibitory capacity, cells growing in flasks were trypsinized with trypsin/EDTA solution (Gibco). The resulting suspension was diluted in complete medium to 1×10⁶ cells/ml. Aliquots of 0.1 ml were pipetted into a 96 well plate in order to screen the large number of antisense sequence derivatives, and concentrations, in triplicate or quadruplicate. The doubling time of OVCAR5 cells is about 3 days, resulting in 2×10⁵ cells/well at the time of analysis, unless proliferation was inhibited.

For transfection of cells, each oligonucleotide (1.0 μM) was mixed with the cationic lipid Lipofectamine PLUS (60 μg/ml) in a low serum medium (Opti-Mem® I) and incubated at room temperature for 30 min. (Wickstrom & Tyson, in Chadwick, D. J., & Cardew, G., eds., Oligonucleotides as Therapeutic Agents, Ciba Foundation Symposium 209, Wiley, Chichester, 124-141, 1997). During this incubation period, the cells were washed in PBS to remove traces of old, complete RPMI medium and resuspended in 0.1 ml of Opti-MEM® I. At the end of the 30-minute incubation, 0.01 ml DNA:lipid coalescence mixture was added to quadruplicate cell samples and incubated at 37° C. for 8 hours. During this incubation, the cells were able to take up the antisense DNAs. The cells then were washed, resuspended in complete RPMI 1640, and incubated at 37° C. for 72 more hours to allow internalized antisense DNAs to bind to target sites on mRNAs, with the subsequent potential to inhibit oncogene expression. In the cases of PNA-peptides, dendrimer-oligonucleotides, and peptide-oligonucleotides, no cationic lipids were necessary to assist uptake.

To measure oncogene antigen production, the treated cells were lysed after incubation, the cell debris was then pelleted, and the supernatants were analyzed for oncogene antigen by Western blotting, relative to actin. (Vaughn, et al. Nucleic Acids Res. 24:4558-4564, 1996; Wickstrom & Tyson, in Chadwick, D. J., & Cardew, G., eds., Oligonucleotides as Therapeutic Agents, Ciba Foundation Symposium 209, Wiley, Chichester, 124-141, 1997; Smith & Wickstrom, J Natl Cancer Inst 90:1146-1154, 1998).

mRNA Levels

Levels of oncogene mRNA, relative to TATA-box binding protein (TBP) or glyceraldehyde phosphate dehydrogenase (GAPDH) mRNA as a control, were measured by Northern blotting (Heikkila et al., Nature 328:445-449, 1987; Vaughn, et al. Nucleic Acids Res. 24:4558-4564, 1996), solution hybridization (Wickstrom, et al., Cancer Res. 52:6741-6745, 1992), or RT/PCR (Kita et al., Int J Cancer 80:553-558, 1999). Parallel untreated cultures of tumor cells, normal cells, and white blood cells were lysed with ToTALLY RNA™ Total RNA Isolation reagents (Ambion, Houston, Tex.). The final pellet was resuspended in 30 μl RNase-free H₂O with RNase inhibitors. The RNA was reverse-transcribed using 50 μg/ml oligo(dT), 500 μM deoxynucleotide triphosphate, and 200 units of Superscript II reverse transcriptase (Life Technologies) for 1 hour at 37° C., and the resulting first strand cDNA was diluted and used as a template for QRT-PCR analysis. Oncogene mRNAs were quantitated with a Prizm 7700 Sequence Detection System (TaqMan) (Applied Biosystems, Foster City, Calif.), which utilizes the 5′ nuclease activity of Taq DNA polymerase to generate a real-time quantitative DNA analysis assay (Holland et al., 1991). A non-extendible oligonucleotide hybridization probe with 5′-fluorescent and 3′-rhodamine (quench) moieties was present during the extension phase of the PCR. Degradation and release of the fluorescent moiety due to the 5′ nuclease activity resulted in peak emission at 518 nm and was monitored every 8.5 seconds by a sequence detector. The increase in fluorescence was monitored during the complete amplification process (real-time). The expression of the housekeeping genes TBP or GAPDH were used to normalize for variances in input cDNA. Primer and fluorescent probe sets calculated using Prizm software for K-RAS (Duffy, unpublished), ERBB2 (Bièche et al., Clin Chem. 45:1148-1156, 1999), and c-MYC (Bièche et al., Cancer Res. 59:2759-2765, 1999) mRNAs were obtained from Applied Biosystems.

Proliferation, Cell Cycle, and Apoptosis

Cell proliferation versus apoptosis was assessed by flow cytometry of propidium iodide stained cells, revealing distribution among G0/G1, S, and G2/M phases; cells with <2n DNA were considered apoptotic. Apoptosis is considered a desirable endpoint of chemotherapy. If a novel antisense DNA is found to be cytotoxic rather than cytostatic for the malignant cells, that oligonucleotide is considered much more valuable for therapy. Cell cycle analysis following propidium iodide staining was used to determine if a subpopulation of viable cells was escaping death by arresting in a particular phase, such as G1. (Heikkila et al., Nature 328:445-449, 1987).

### EXAMPLE 2

### In Vivo Anti Tumorigenesis

Malignant Cell Xenografts

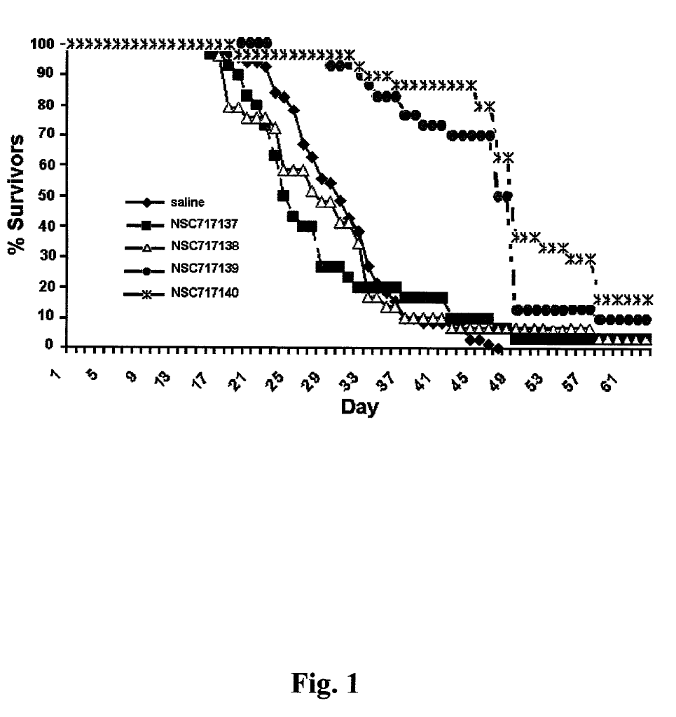

Potency against tumorigenesis and reduction of oncogene expression in malignant human OVCAR5 ovarian tumor cell xenografts in 6-8 week old female Balb/c nu/nu immunocompromised mice was measured. In groups of 10 subjects, aliquots of 1×10⁶ malignant cells were implanted subcutaneously by sterile 25 gauge syringe into the flank of each subject. The subcutaneous site yielded a localized, measurable, recoverable tumor, as opposed to the disseminated ascites model utilized in the OVCAR5 challenge experiments below (FIG. 1). In case of pain or distress from tumor cell implantation, the subjects were supplied analgesics in the form of a tylenol-codeine elixir mixed 3 ml to 250 ml H₂O, administered ad libitum in the drinking water.

Oligonucleotide Administration

The antisense oligonucleotides were administered intraperitoneally for up to four (4) weeks, and assessed for their effects on tumor growth, oncogene expression, cell cycle distribution, or apoptosis. Oligonucleotides were administered intraperitoneally daily, or every other day, at concentrations of 1-20 mg/kg for 1-4 weeks before or after establishment of palpable tumors. In the case of orally available derivatives, the oligonucleotides were administered in sterile drinking water at 1-20 mg/kg.

Assessment of Efficacy

Each experiment had an endpoint of 60 days past the initial observation of palpable tumor, unless animal morbidity deemed early termination. Animals that become moribund, lethargic, or anorexic were euthanized. Animals whose tumors exceeded 2 cm³, grew into the body cavity, or showed ulceration also were euthanized. At the conclusion of each experiment, after euthanasia by cervical dislocation or CO₂ inhalation, xenograft tumors were removed from the animals in each treatment group for tumor volume (l×w²/2) or mass measurement, histopathological evaluation, biochemical analysis of antigen and mRNA levels, and detection of apoptosis, as in the cell culture experiments.

Survival times were measured in groups of 10 nude female mice (20 for vehicle control) implanted intraperitoneally with 10⁶ human OVCAR5 ovarian cancer cells. Each mouse was administered 1 mg of oligonucleotide intraperitoneally every other day for 14 days, with the first dose administered one day following tumor inoculation. Three independent trials were carried out.

For comparison, pairs of mice were treated on days 1, 5, and 9 after OVCAR5 implantation with methotrexate, actinomycin D, chlorambucil, L-PAM, 5-FU, cyclophosphamide, mitomycin C, DTIC, vinblastine, adriamycin, BCNU, cisplatin, paclitaxel, and bleomycin.

In a third trial, to control for K-RAS oncogene activation, OVCAR3 cells were implanted into three other groups of nude mice. OVCAR3 cells do not display K-RAS oncogene activation. One group of mice received vehicle, one group received NSC71739, and the third received NSC717140.

Metastasis

Intraperitoneal growth and dissemination of tumor cell xenografts with malignant OVCAR5 cancer cells has been observed. The sites of intraperitoneal xenograft dissemination in mice correspond to sites of metastatic tumor growth observed clinically, i.e., the mesentery, pancreas, and the hepatic hilus. To measure treatment of this model of malignancy, 1×10⁶ malignant cells were injected intraperitoneally into each of 10 mice per treatment group, as well as the PBS vehicle control group. Efficacy of treatment was determined by size and number of lesions scored on the mesentery, pancreas, and the hepatic hilus.

Oligonucleotide Modifications

Antisense compounds used in the invention also may include chimeric oligonucleotides or chimeras. In the context of this invention, chimeras or chimeric oligonucleotides are oligonucleotides that contain two or more chemically distinct regions, each made up of at least one nucleotide. These oligonucleotides typically contain at least one pendant group or moiety, either as part of or separate from the basic repeat unit of the polymer, to enhance specificity, nuclease resistance, delivery, or other properties related to efficacy, e.g., peptide analogs, cholesterol moieties, duplex intercalators such as acridine, poly-L-lysine, “end-capping” with one or more nuclease-resistant linkage groups such as phosphorothioate, and the like. Antisense oligonucleotides of the invention also may contain a region that is a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of antisense inhibition of gene expression. Cleavage of the mRNA target can be routinely detected by gel electrophoresis and, if necessary, associated nucleic acid hybridization techniques known in the art.

By way of further example, it is known that enhanced lipid solubility and/or resistance to nuclease digestion results by substituting an alkyl group, alkoxy group, or borano group in place of a phosphate oxygen in the internucleotide phosphodiester linkage to form an alkylphosphonate oligonucleoside, alkylphosphotriester oligonucleotide, or borane phosphate oligonucleotide. Non-ionic oligonucleotides such as these are characterized by increased resistance to nuclease hydrolysis, while retaining the ability to form stable complexes with complementary nucleic acid sequences. The alkylphosphonates, in particular, are stable to nuclease cleavage and soluble in lipid. The preparation of alkylphosphonate oligonucleosides is disclosed in Ts'o et al., U.S. Pat. No. 4,469,863, herein incorporated by reference. The preparation of stereoregular alkylphosphonate oligonucleosides is disclosed in Wickstrom & Le Bec, U.S. Pat. No. 5,703,223, herein incorporated by reference.

Preferably, nuclease resistance is conferred on the antisense compounds of the invention by providing nuclease-resistant internucleosidic linkages. Many such linkages are known in the art, e.g., phosphorothioate: Zon & Geiser, Anti-Cancer Drug Design, 6:539-568, 1991; Stec et al., U.S. Pat. No. 5,151,510; Hirschbein, U.S. Pat. No. 5,166,387; Bergot, U.S. Pat. No. 5,183,885; phosphorodithioates: Marshall et al., Science 259:1564-1570, 1993; Caruthers & Nielsen, International application PCT/US89/02293; phosphoramidates: Jager et al., Biochemistry 27:7237-7246, 1988; Froehler et al., International application PCT/US90/03138; peptide nucleic acids: Nielsen et al., Anti-Cancer Drug Design 8:53-63, 1993, International application PCT/EP92/01220; methylphosphonates: Miller et al., U.S. Pat. No. 4,507,433; Ts'o et al., U.S. Pat. No. 4,469,863; Miller et al., U.S. Pat. 4,757,055; borane phosphates: Spielvogel et al., U.S. Pat. No. 5,859,231; and P-chiral linkages of various types, especially phosphorothioates, Stec et al., European patent application 506,242 (1992) and Lesnikowski, Bioorganic Chemistry 21:127-155, 1993. Additional nuclease linkages include phosphoroselenoate, phosphorodiselenoate, phosphoroanilothioate, phosphoranilidate, alkylphosphotriester such as methyl- and ethylphosphotriester, carbonate such as carboxymethyl ester, carbamate, morpholino phosphorodiamidate, 3′-thioformacetal, silyl such as dialkyl (C₁-C₆)- or diphenylsilyl, sulfamate ester, and the like. Such linkages and methods for introducing them into oligonucleotides are described in many references, see e.g., reviewed generally by Wickstrom, Prospects for Antisense Nucleic Acid Therapy of Cancer and AIDS, Wiley-Liss, New York (1991); Wickstrom, Trends In Biotechnology, 10:281-287, 1992; Milligan et al., J. Med. Chem. 36:1923-1937, 1993; and Matteucci et al., International application PCT/US91/06855.

Resistance to nuclease digestion may also be achieved by modifying the internucleotide linkage at both the 5′ and 3′ termini with phosphoramidates according to the procedure of Dagle et al., Nucl. Acids Res. 18, 4751-4757, 1990.

Oligonucleotides used in the present invention also may contain one or more substituted sugar moieties. Preferred oligonucleotides comprise one of the following at the 2′ position: OH, SH, SCH₃, F, OCN, OCH₃OCH₃, OCH₃O(CH₂)ₙCH₃, O(CH₂)ₙ NH₂ or O(CH₂)ₙCH₃ where n is from 1 to about 10; C₁ to C₁₀ lower alkyl, alkylalkoxy, substituted lower alkyl, aryl or alkaryl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a receptor ligand analog; a group for improving the pharmacokinetic properties of an oligonucleotide; or a group for improving the pharmacodynamic properties of an oligonucleotide, and other substituents having similar properties. A preferred modification includes 2′-methoxyethoxy [2′-O-CH₂CH₂0CH₃, also known as 2′-O-(2-methoxyethyl)] (Martin et al., Helv. Chim. Acta 78:486, 1995). Other preferred modifications include 2′-methoxy (2′-O-CH₃), 2′-propoxy (2′-OCH₂CH₂CH₃) and 2′-fluoro (2′-F). Similar modifications also may be made at other positions on the oligonucleotide, particularly the 3′ position of the sugar on the 3′ terminal nucleotide and the 5′ position of the 5′ terminal nucleotide. Oligonucleotides also may have sugar mimetics such as cyclobutyls in place of the pentofuranosyl group.

The oligonucleotides of the invention may be provided as prodrugs, which comprise one or more moieties that are cleaved off, generally in the body, to yield an active oligonucleotide. One example of a prodrug approach is described by Imbach et al. in WO Publication 94/26764 and Vives, et al. Nucleic Acids Res. 20:4071-4076, 1999.

Oligonucleotides for use in the present invention also may include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-methyl pyrimidines, particularly 5-methylcytosine (also referred to as 5-methyl-2′-deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N⁶(6-aminohexyl)adenine and 2,6-diaminopurine. (Kornberg, A., DNA Replication, W. H. Freeman & Co., San Francisco, 75-77 (1980); Gebeyehu, G., et al., Nucleic Acids Res. 15:4513, 1987). A “universal” base known in the art, e.g., inosine, may be included. 5-Me-C substitutions have been shown to increase nucleic acid duplex stability by 0.6-1.2° C. (Sanghvi, Y. S., in Crooke, S. T. & Lebleu, B., eds., Antisense Research and Applications, CRC Press, Boca Raton, 276-278 (1993)) and are presently preferred base substitutions.

Oligonucleotides envisioned for this invention include those containing modified backbones, for example, phosphorothioates, borane phosphates, phosphotriesters, methyl phosphonates, short chain alkyl or cycloalkyl intersugar linkages or short chain heteroatomic or heterocyclic intersugar linkages. Preferably, phosphorus analogs of the phosphodiester linkage are employed in the compounds of the invention, such as phosphorothioate, phosphorodithioate, phosphoramidate, or methylphosphonate. More preferably, phosphorothioate is employed as the nuclease resistant linkage. Phosphorothioate oligonucleotides contain a sulfur-for-oxygen substitution in the internucleotide phosphodiester bond. Phosphorothioate oligonucleotides combine the properties of effective hybridization for duplex formation with substantial nuclease resistance, while retaining the water solubility of a charged phosphate analogue. The charge, like that on a normal phosphodiester, is believed to confer the property of cellular uptake via a receptor. (Yakubov et al., Proc. Natl. Acad. Sci. USA 86:6454-6458, 1989).

Amide backbones disclosed by De Mesmaeker et al. (Acc. Chem. Res. 28:366-374, 1995) also are preferred. Also preferred are oligonucleotides having morpholino backbone structures. (Summerton & Weller, U.S. Pat. No. 5,034,506). In other embodiments, the phosphodiester backbone of the oligonucleotide is replaced with a polyamide backbone, the nucleobases being bound directly or indirectly to the aza nitrogen atoms of the polyamide backbone. (Nielsen et al. Science 254, 1497-1499, 1991).

It is understood that in addition to the preferred linkage groups, the oligonucleotides used in the invention may comprise additional modifications, e.g., boronated bases, Spielvogel et al., U.S. Pat. No. 5,130,302; and cholesterol moieties, Shea et al., Nucleic Acids Research 18:3777-3783, 1990; or Letsinger et al., Proc. Natl. Acad. Sci. 86:6553-6556, 1989.

Oligonucleotide Synthesis

Antisense compounds of the invention can be synthesized by conventional means on commercially available automated DNA synthesizers, e.g., an Applied Biosystems (Foster City, Calif.) synthesizer. Preferably, phosphoramidite chemistry is employed, e.g., as disclosed in the following references: Beaucage & Iyer, Tetrahedron 48:2223-2311, 1992; Molko et al., U.S. Pat. No. 4,980,460; Köster et al., U.S. Pat. No. 4,725,677; and Caruthers et al., U.S. Pat. Nos. 4,415,732; 4,458,066; and 4,973,679. Any other means for such synthesis also may be employed. It also is well known to use similar techniques to prepare other oligonucleotides, such as the phosphorothioates and alkylated derivatives. It also is well known to use similar techniques and commercially available modified amidites and controlled-pore glass (CPG) products such as biotin, fluorescein, acridine or psoralen-modified amidites and/or CPG (Glen Research, Sterling, Va.) to synthesize fluorescently labeled, biotinylated, or other modified oligonucleotides such as cholesterol-modified oligonucleotides.

Pharmaceutical Compositions

Pharmaceutical compositions of the invention include a pharmaceutical carrier that may contain a variety of components that provide a variety of functions, including regulation of drug concentration, regulation of solubility, chemical stabilization, regulation of viscosity, absorption enhancement, regulation of pH, and the like. The pharmaceutical carrier may comprise a suitable liquid vehicle or excipient and an optional auxiliary additive or additives. The liquid vehicles and excipients are conventional and commercially available. Illustrative thereof are distilled water, physiological saline, aqueous solutions of dextrose, and the like. For water-soluble formulations, the pharmaceutical composition preferably includes a buffer such as a phosphate buffer, or other organic acid salt, preferably at a pH of between about 7 and 8. For formulations containing weakly soluble antisense compounds, micro-emulsions may be employed, for example by using a nonionic surfactant such as polysorbate 80 in an amount of 0.04-0.05% (w/v), to increase solubility. Other components may include antioxidants, such as ascorbic acid, hydrophilic polymers, such as, monosaccharides, disaccharides, and other carbohydrates including cellulose or its derivatives, dextrins, chelating agents, such as EDTA, and like components well known to those in the pharmaceutical sciences.

Antisense compounds of the invention include the pharmaceutically acceptable salts thereof, including those of alkaline earths, e.g., sodium or magnesium, ammonium or NX4.⁺, wherein X is C₁-C₄ alkyl. Pharmaceutically acceptable salts of a compound having a hydroxyl group include the anion of such compound in with a suitable cation such as Na⁺, NH₄⁺, or the like.

Administration

The antisense oligonucleotides are preferably administered parenterally, most preferably intravenously. The vehicle is designed accordingly. Alternatively, oligonucleotide may be administered subcutaneously via controlled release dosage forms. In view of the oral availability of mixed backbone oligonucleotides (Agrawal, et al. Biochem. Pharmacol. 50:571-576, 1995), enteric-coated tablets would be suitable for oral administration.

In addition to administration with conventional carriers, the antisense oligonucleotides may be administered by a variety of specialized oligonucleotide delivery techniques. Sustained release systems suitable for use with the pharmaceutical compositions of the invention include semi-permeable polymer matrices in the form of films, microcapsules, or the like, comprising polylactides, copolymers of L-glutamic acid and gamma-ethyl-L-glutamate, poly(2-hydroxyethyl methacrylate), and like materials, e.g., Rosenberg et al., International application PCT/US92/05305.

For systemic or regional in vivo administration, the amount of antisense oligonucleotides may vary depending on the nature and extent of the neoplasm, the particular oligonucleotides utilized, and other factors. The actual dosage administered may take into account the size and weight of the patient, whether the nature of the treatment is prophylactic or therapeutic in nature, the age, health and sex of the patient, the route of administration, whether the treatment is regional or systemic, and other factors. Intercellular concentrations of therapeutic oligonucleotide from about 0.1 to about 20 μg/ml in the target tissue may be employed, preferably from about 10 μg/ml to about 100 μg/ml intracellularly at the target polynucleotide. The patient should receive a sufficient daily dosage of antisense oligonucleotide to achieve the desired intercellular tissue concentrations of combined oligonucleotides. The daily oligonucleotide dosage may range from about 25 mg to about 2 grams per day, with at least about 250 mg being preferred. Greater or lesser amounts of oligonucleotide may be administered, as required. Those skilled in the art should be readily able to derive appropriate dosages and schedules of administration to suit the specific circumstance and needs of the patient. For modified oligonucleotides, such as phosphorothioate oligonucleotides, which have a half life of from 24 to 48 hours, the treatment regimen may comprise dosing on alternate days.

The antisense oligonucleotides of the instant invention may be used as the primary therapeutic for the treatment of the disease state, or may be used in conjunction with non-oligonucleotide agents.

Therapeutic Delivery

Antisense compounds of the present invention include conjugates of such oligonucleotides with appropriate ligand-binding molecules. The oligonucleotides may be conjugated for therapeutic administration to ligand-binding molecules that recognize cell-surface molecules. The ligand-binding molecule may comprise, for example, an antibody against a cell surface antigen, an antibody against a cell surface receptor, a growth factor having a corresponding cell surface receptor, an antibody to such a growth factor, or an antibody that recognizes a complex of a growth factor and its receptor. Methods for conjugating ligand-binding molecules to oligonucleotides are known in the art, e.g., Basu & Wickstrom, U.S. Pat. No. 6,180,767, incorporated herein by reference.

Gene Therapy

As an alternative to treatment with exogenous oligonucleotides, antisense polynucleotide synthesis may be induced in situ by local treatment of the targeted neoplastic cells with a vector containing an artificially-constructed gene comprising transcriptional promoters and targeted oncogene DNA in inverted orientation to allow antisense transcription. The DNA for insertion into the artificial gene in inverted orientation comprises cDNA that may be prepared, for example, by reverse transcriptase polymerase chain reaction from RNA using primers derived from the published target oncogene cDNA sequences.

A first DNA segment for insertion contains cDNA of a cytoplasmic oncogene. A second DNA segment for insertion contains cDNA of a nuclear oncogene. The two segments are under control of corresponding first and second promoter segments. Upon transcription, the inverted oncogene segments, which are complementary to the corresponding targeted oncogene, are produced in situ in the targeted cell. The endogenously produced RNAs hybridize to the relevant oncogene mRNAs, resulting in interference with oncogene function and inhibition of the proliferation of the targeted cell.

The promoter segments of the artificially-constructed gene serve as signals conferring expression of the inverted oncogene sequences that lie downstream thereof. Each promoter will include all of the signals necessary for initiating transcription of the relevant downstream sequence. Each promoter may be of any origin as long as it specifies a rate of transcription that will produce sufficient antisense mRNA to inhibit the expression of the target oncogene and, therefore, the proliferation of the targeted cells. Preferably, a highly efficient promoter such as a viral promoter is employed. Other sources of potent promoters include cellular genes that are expressed at high levels. The promoter segment may comprise a constitutive or a regulatable promoter.

The artificial gene may be introduced by any of the methods described in U.S. Pat. No. 4,740,463, incorporated herein by reference. One technique is transfection, which can be done by several different methods, including phospholipid-mediated delivery (supra). In particular, polycationic liposomes can be formed from N-1-(2,3-di-oleyloxy) propyl-N,N,N-trimethylammonium chloride (DOTMA). See Felgner et al., Proc. Natl. Acad. Sci. USA 84, 7413-7417, 1987 (DNA-transfection); and Malone et al., Proc. Natl. Acad. Sci. USA 86, 6077-6081, 1989 (RNA-transfection). Vesicle fusion also could be employed to deliver the artificial gene. Vesicle fusion may be physically targeted to the malignant cells if the vesicles are designed to be taken up by those cells. Such a delivery system would be expected to have a lower efficiency of integration and expression of the artificial gene delivered, but would have a higher specificity than a retroviral vector. A strategy of targeted vesicles containing papilloma virus or retrovirus DNA molecules might provide a method for increasing the efficiency of expression of targeted molecules.

Alternatively, the artificially-constructed gene can be introduced into cells, in vitro or in vivo, via a transducing viral vector. Use of a retrovirus, for example, will infect a variety of cells and cause the artificial gene to be inserted into the genome of infected cells. Such infection could either be accomplished with the aid of a helper retrovirus, which would allow the virus to spread through the organism, or the antisense retrovirus could be produced in a helper-free system. A helper-free virus might be employed to minimize spread throughout the organism. Viral vectors in addition to retroviruses also can be employed, such as papovaviruses, SV40-like viruses, or papilloma viruses.

Particulate systems and polymers for in vitro and in vivo delivery of polynucleotides were extensively reviewed by Felgner in Advanced Drug Delivery Reviews 5:163-187, 1990. Techniques for direct delivery of purified genes in vivo, without the use of retroviruses, has been reviewed by Felgner in Nature 349:351-352, 1991. Such methods of direct delivery of polynucleotides may be utilized for local delivery of either exogenous oncogene antisense oligonucleotide or artificially-constructed genes producing oncogene antisense oligonucleotide in situ.

Nonviral, site-specific transposition of precisely one antisense gene per haploid genome may be utilized, using the method of Cleaver & Wickstrom, U.S. Pat. No. 5,958,775, incorporated herein by reference.

Efficacy

The effectiveness of the treatment may be assessed by routine methods that are used for determining whether or not remission has occurred. Such methods generally depend upon some morphological, cytochemical, cytogenetic, immunologic and/or molecular analyses. In addition, remission can be assessed genetically by probing the level of expression of one or more relevant oncogenes. The reverse transcriptase polymerase chain reaction methodology can be used to detect even very low numbers of mRNA transcripts.

Typically, therapeutic success is assessed by the decrease and the extent of the primary, and any metastatic, disease lesions. For solid tumors, decreasing tumor size is the primary indicia of successful treatment. Neighboring tissues should be biopsied to determine the extent to which metastasis has occurred. Tissue biopsy methods are known to those skilled in the art.

Results

As noted above, efficacy of intraperitoneal oligonucleotide treatment was assessed by measuring survival times in groups of 10 nude female mice (20 for vehicle control) implanted intraperitoneally with 10⁶ human OVCAR5 ovarian cancer cells. (FIG. 1) The results demonstrate that powerful protection is provided not only by NSC717139 but also by NSC717140, with four central mismatches. Only small lumps at the site of cell injection were observed in the protected mice, while the mice treated with saline vehicle, NSC717137, and NSC717138 swelled up rapidly with ascites, killing both cohorts entirely by 5 weeks. The mice protected by NSC717139 and NSC717140 showed evidence of disease at death, suggesting that those mice did not die due to the effects of the oligonucleotides. In comparison, examination of mice treated on days 1, 5, and 9 after OVCAR5 implantation with methotrexate, actinomycin D, chlorambucil, L-PAM, 5-FU, cyclophosphamide, mitomycin C, DTIC, vinblastine, adriamycin, BCNU, cisplatin, paclitaxel and bleomycin revealed that, with the exception of paclitaxel and bleomycin, these agents are inactive in 1/2 or 2/2 tests conducted.

To control for K-RAS oncogene activation, OVCAR3 cells were implanted into three groups of nude mice: one group received vehicle, one group received NSC71739, and the third received NSC717140. OVCAR3 cells do not display K-RAS oncogene activation. No increased survival was seen in any group. These results imply that the effects of NSC717139 and NSC717140 are limited to ovarian cancer cells transformed by mutant K-RAS oncogene.

What is claimed is:

1. A method of preventing or treating cancer in a mammal suspected of having cancer comprising
a) administering to said mammal suspected of having cancer a therapeutically effective amount of an oligonucleotide that is targeted to a nucleic acid encoding human K-RAS oncogene wherein said oligonucleotide comprises SEQ. ID. NO: 1, 2, 6, or 7;
b) modulating K-RAS expression; and
c) preventing or treating said cancer.

2. The method of claim 1 wherein said mammal is a human.

3. A method of preventing or treating cancer, wherein said cancer is at least one of the group of colon cancer, hematologic cancer, lung cancer, ovarian cancer, prostate cancer, skin cancer, and thyroid cancer, in a mammal comprising
b) administering to said mammal suspected of having cancer a therapeutically effective amount of an oligonucleotide that is targeted to a nucleic acid encoding human K-RAS oncogene wherein said oligonucleotide comprises SEQ. ID. NO: 1, 2, 6 or 7;
b) modulating K-RAS expression; and
c) preventing or treating said cancer.

4. The method of claim 3 wherein said mammal is a human.

5. A method of preventing or treating ovarian cancer in a mammal comprising
d) administering to said mammal suspected of having cancer a therapeutically effective amount of an oligonucleotide that is targeted to a nucleic acid encoding human K-RAS oncogene wherein said oligonucleotide comprises SEQ. ID. NO: 1, 2, 6 or 7;
b) modulating K-RAS expression; and
c) preventing or treating said cancer.

6. The method of claim 5 wherein said mammal is a human.

7. A pharmaceutical composition for the treatment of cancer comprising an oligonucleotide that is targeted to a nucleic acid encoding human K-RAS oncogene wherein said oligonucleotide comprises SEQ. ID. NO: 1, 2, 6 or 7.

8. A pharmaceutical composition for the treatment of cancer, wherein said cancer is at least one of the group of colon cancer, hematologic cancer, lung cancer, ovarian cancer, prostate cancer, skin cancer, and thyroid cancer, comprising an oligonucleotide that is targeted to a nucleic acid encoding human K-RAS oncogene wherein said oligonucleotide comprises SEQ. ID. NO: 1, 2, 6 or 7

9. A pharmaceutical composition for the treatment of ovarian cancer comprising an oligonucleotide that is targeted to a nucleic acid encoding human K-RAS oncogene wherein said oligonucleotide comprises SEQ. ID. NO: 1, 2, 6 or 7.

10. A method of inhibiting the proliferation of cancer cells comprising contacting said cancer cells with an effective amount of an oligonucleotide comprising SEQ. ID. NO: 1, 2, 6 or 7, whereby proliferation of the cancer cells is inhibited.

11. A method of inhibiting the proliferation of cancer cells, wherein said cancer cells are at least one of the group of colon cancer cells, hematologic cancer, lung cancer cells, ovarian cancer cells, prostate cancer cells, skin cancer cells, and thyroid cancer cells, comprising contacting said cancer cells with an effective amount of an oligonucleotide comprising SEQ. ID. NO: 1, 2, 6 or 7, whereby proliferation of the cancer cells is inhibited.

12. A method of inhibiting the proliferation of ovarian cancer cells comprising contacting said cancer cells with an effective amount of an oligonucleotide comprising SEQ. ID. NO: 1, 2, 6 or 7, whereby proliferation of the cancer cells is inhibited.

13. A method of modulating the expression of mutation-activated K-RAS oncogene in tissues or cells containing a mutation-activated K-RAS oncogene comprising contacting said tissues or cells containing a mutation-activated K-RAS oncogene with an effective amount of an oligonucleotide comprising SEQ. ID. NO: 1, 2, 6 or 7 whereby expression of mutation-activated K-RAS is modulated.

14. A method of modulating the expression of mutation-activated K-RAS oncogene in tissues or cells containing a mutation-activated K-RAS oncogene, wherein said tissues or cells containing a mutation-activated K-RAS oncogene are at least one of the group of colon cancer cells, hematologic cancer, lung cancer cells, ovarian cancer cells, prostate cancer cells, skin cancer cells, and thyroid cancer cells, comprising contacting said tissues or cells containing a mutation-activated K-RAS oncogene with an effective amount of an oligonucleotide comprising SEQ. ID. NO: 1, 2, 6 or 7 whereby expression of mutation-activated K-RAS is modulated.

15. A method of modulating the expression of mutation-activated K-RAS oncogene in ovarian tissues or cells containing a mutation-activated K-RAS oncogene comprising contacting said ovarian tissues or cells containing a mutation-activated K-RAS oncogene with an effective amount of an oligonucleotide comprising SEQ. ID. NO: 1, 2, 6 or 7 whereby expression of mutation-activated K-RAS is modulated.

16. A method of preventing or treating a condition arising from the activation of a K-RAS oncogene in a mammal suspected of having a condition arising from the activation of a K-RAS oncogene comprising
a) administering to said mammal suspected of having a condition arising from the activation of a K-RAS oncogene an effective amount of an oligonucleotide that is targeted to a nucleic acid encoding human K-RAS oncogene wherein said oligonucleotide comprises SEQ. ID. NO: 1, 2, 6or 7;
b) modulating K-RAS expression; and
c) preventing or treating said condition.

17. The method of claim 16 wherein said mammal is a human.

18. An oligonucleotide 8 to 15 nucleotides in length which is targeted to a nucleic acid encoding human K-RAS and which is capable of modulating K-RAS expression, wherein said oligonucleotide is complemenatary to a region between nucleotides 138 and 147 of human K-RAS.

19. The oligonucleotide of claim 18, wherein said oligonucleotide comprises SEQ. ID. NO: 6 or 7.

20. The oligonucleotide of claim 18 which comprises at least one backbone modification.

21. The oligonucleotide of claim 18 wherein at least one of the nucleotide units of the oligonulceotide is modified at the 2′ position of the sugar.

22. The oligonucleotide of claim 18 which is a chimeric oligonucleotide.

23. The oligonucleotide of claim 18 in a pharmaceutically acceptable carrier.


SEQUENCE LISTING (7 sequences)

SEQ ID NO: 1 - DNA, 15 bp, Artificial Sequence
  Synthetic Oligonucleotide
agtcgccccgccgca

SEQ ID NO: 2 - DNA, 15 bp, Artificial Sequence
  Synthetic Oligonucleotide
agtcgaaaagccgca

SEQ ID NO: 3 - DNA, 15 bp, Artificial Sequence
  Synthetic Oligonucleotide
ggtgctcactgcggc

SEQ ID NO: 4 - DNA, 15 bp, Artificial Sequence
  Synthetic Oligonucleotide
ggtgcagtgtgcggc

SEQ ID NO: 5 - DNA, 10 bp, Artificial Sequence
  Synthetic Oligonucleotide
gcggcggggc

SEQ ID NO: 6 - DNA, 10 bp, Artificial Sequence
  Synthetic Oligonucleotide
gccccgccgc

SEQ ID NO: 7 - DNA, 10 bp, Artificial Sequence
  Synthetic Oligonucleotide
gaaaagccgc